(19) United States
(12) Patent Application Publication
(10) Pub. No.: US 2012/0122973 A1
(43) Pub. Date: May 17, 2012

(54) DRY PROCESSING OF RETIGABINE

(75) Inventors: Jana Paetz, Bonn (DE); Katrin Rimkus, North Rhine-Westphalia (DE)

(73) Assignee: ratiopharm GmbH, Ulm, Baden-Wurttemberg (DE)

(21) Appl. No.: 13/257,056

(22) PCT Filed: Mar. 17, 2010
(86) PCT No.: PCT/EP2010/001690

§ 371 Date: Oct. 31, 2011

(30) Foreign Application Priority Data
Mar. 17, 2009 (DE) 10 2009 013 613.4

(51) Int. Cl. A61K 31/27; A61P 25/04; A61K 9/28; A61P 25/08

(52) U.S. Cl. 514/485; 427/2.14

(57) ABSTRACT

The invention relates to dry processes for the production of oral dosage forms, especially tablets, containing retigabine and adhesion promoter. In addition, the invention relates to compacted intermediates containing retigabine and an adhesion promoter. Finally, the invention relates to single-dose and multiple-dose containers, preferably sachets and stick-packs, containing the intermediate of the invention.

The invention relates to dry processes for the production of oral dosage forms, especially tablets, containing retigabine and adhesion promoter. In addition, the invention relates to compacted intermediates containing retigabine and an adhesion promoter.

The IUPAC name of retigabine [INN] is 2-amino-4-(4-fluorobenzylamino)-1-ethoxy-carbonyl aminobenzene. The chemical structure of retigabine is shown in formula (1) below:

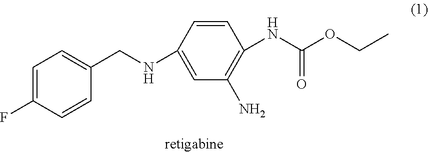

Synthesis pathways for retigabine and its use as an anti-epileptic agent have been described in EP 0 554 543. The use of retigabine for the treatment of neuropathic pain is also known from WO 01/22953 A2.

Epilepsy is one of the commonest neurological disorders and affects up to about 1% of the population. Whereas a majority of epilepsy patients can be treated with anticon-vulsants currently available on the market, about 30% of patients are pharmaco-resistant. There is therefore a need to develop new anticonvulsants with innovative mechanisms of action. As a potassium channel opener, retigabine, an anticonvulsant substance, satisfies these criteria. As yet, however, no pharmaceutical dosage forms are known in the art which permit an advantageous, oral administration of retigabine in high doses, especially with modified release, for the treatment of epilepsy.

WO 02/80898 A2 proposes formulating retigabine in the form of hard gelatine capsules containing 50, 100 and 200 mg active agent. Hard gelatine capsules are often felt by patients to be unpleasant to take. In particular, it is problematic to obtain a high content of active agent (e.g. 70%) in the capsule with this method. It has also become apparent that capsules produced by means of the wet granulation of retigabine are not ideal with regard to their pharmacokinetic properties.

In addition, retigabine formulations are proposed in WO 01/66081 A2 which were produced by melt granulation, where a composition consisting solely of retigabine and sucrose fatty acid ester was used. The use of large amounts of sucrose fatty acid ester is often undesirable, however, because of the emulsifier effect. Furthermore, the formulations proposed merely permit delayed release.

The object of the present invention was therefore to overcome the above-mentioned disadvantages.

One object of the invention is to provide a dosage form which is pleasant for the patients and which makes it possible also to administer amounts of active agent of considerably more than 200 mg in an advantageous manner.

The intention is to provide the active agent in a form which possesses good flowability—despite any possible micronisation—and makes good compression possible. The resulting tablets should exhibit a high level of hardness and low friability.

In particular, it is an object of the invention to provide a process for the production of tablets containing retigabine which exhibit advantageous lacquer coatability. During lacquer coating of the tablets of the invention, it is intended that no spalling should occur.

The intention is likewise to provide a granule formulation of retigabine which can advantageously be used in the production of a suspension to be swallowed. The granules should flow well, not separate during storage, and enable exact dosaging from single-dose and multiple-dose containers.

While developing retigabine formulations, the inventors of the present application were also confronted with the fact that crystalline retigabine can exist in different polymorphous forms. As described in WO 98/31663, these polymorphs are frequently not stable, however, but tend to change into different polymorphous forms. The frequently used retigabine form A, for example, can change into form B under the influence of heat. However, the polymorphous forms A, B and C have different solubility profiles.

In a patient, the different solubility profile leads to an undesirable, uneven rise in the concentration of the active agent. It is therefore an object of the present invention to provide stable retigabine intermediates that can be processed into a dosage form which enables as even a rise as possible in the concentration in the patient. The aim is largely to avoid both inter-individual and also intra-individual deviations.

The intention is also to provide dosage forms of retigabine which ensure good solubility and bioavailability with good storage stability at the same time.

All the objects mentioned above are supposed to be achieved in particular for a high content of active agent (drug load).

It has unexpectedly been possible to solve the problems by means of the dry processing of retigabine together with an adhesion promoter.

One subject matter of the invention is therefore a process for the production of oral dosage forms, especially tablets containing retigabine and adhesion promoter, wherein the oral dosage forms, especially the tablets, are produced by means of dry granulation or by means of direct compression: A further subject matter of the invention is tablets which are obtainable by means of the embodiments of the process of the invention described below.

An intermediate obtainable by jointly dry-compacting retigabine with an adhesion promoter is a further subject matter of the invention.

Finally, another subject matter of the invention is single-dose and multiple-dose containers, preferably sachets and stick-packs containing the intermediate of the invention.

In the context of this invention, the term “retigabine” comprises 2-amino-4-(4-fluoro-benzylamino)-1-ethoxycarbonyl aminobenzene according to the above formula (1). In addition, the term “retigabine” comprises all the pharmaceutically acceptable salts, hydrates and solvates thereof.

The salts may be acid addition salts. Examples of suitable salts are hydrochlorides (monohydrochloride, dihydrochloride), carbonates, hydrogen carbonates, acetates, lactates, butyrates, propionates, sulphates, methane sulphonates, citrates, tartrates, nitrates, sulphonates, oxalates and/or succinates. Retigabine is preferably used in the form of the free base or in the form of dihydrochloride.

In the context of this invention, retigabine can be used both in amorphous and in crystalline form. Similarly, retigabine can also be used in the form of a solid solution

According to WO 98/31663, crystalline retigabine may be present in three different polymorphous forms (polymorphous forms A, B and C). In the context of this invention, in the case of crystalline retigabine, the polymorphous form A is preferably used.

The adhesion promoter is generally a substance which is suitable for stabilising retigabine in compacted or compressed form. The addition of the adhesion promoter usually leads to an increase in the size of the interparticulate surfaces, where bonds can form (e.g. during the compression process). In addition, adhesion promoters are characterised by the fact that they increase the plasticity of the tableting mixture, so that solid tablets form during compression.

In one possible embodiment, the adhesion promoter is a polymer. In addition, the term “adhesion promoter” also includes substances which behave like polymers. Examples of these are fats and waxes, but not sucrose fatty acid ester. Furthermore, the adhesion promoter also includes solid, non-polymeric compounds which preferably contain polar side groups. Examples of these are sugar alcohols or disaccharides.

The adhesion promoter used in the context of this invention is preferably a polymer which has a glass transition temperature (Tg) higher than 15° C., more preferably 40° C. to 150° C., especially 50° C. to 100° C.

The term “glass transition temperature” (Tg) is used to describe the temperature at which amorphous or partially crystalline polymers change from the solid state to the liquid state. In the process, a distinct change in physical parameters, e.g. hardness and elasticity, occurs. Below the Tg, a polymer is usually glassy and hard, whereas above the Tg, it changes into a rubber-like to viscous state. The glass transition temperature is determined in the context of this invention by means of dynamic differential scanning calorimetry (DSC). For this purpose, a Mettler Toledo DSC 1 apparatus, for example, can be used. The work is performed at a heating rate of 1-20° C./min, preferably 5-15° C./min, and at a cooling rate of 5-25, preferably 10-20° C./min.

In addition, the polymer which can be used as an adhesion promoter preferably has a weight-average or number-average molecular weight of 1,000 to 500,000 g/mol, more preferably 2,000 to 90,000 g/mol. When the polymer used to produce the intermediate is dissolved in water in an amount of 2% by weight, the resulting solution preferably has a viscosity of 0.1 to 8 mPa×s, more preferably 0.3 to 7 mPa×s, especially 0.5 to 4 mPa×s, measured at 25° C. and preferably determined in accordance with Ph. Eur., 6th edition, chapter 2.2.10.

Hydrophilic polymers are preferably used for the preparation of the intermediate. This refers to polymers which possess hydrophilic groups. Examples of suitable hydrophilic groups are hydroxy, alkoxy, acrylate, methacrylate, sulphonate, carboxylate and quaternary ammonium groups.

The intermediate of the invention may, for example, comprise the following polymers as adhesion promoters: polysaccharides, such as hydroxypropyl methyl cellulose (HPMC), carboxymethyl cellulose (CMC, especially sodium and calcium salts), ethyl cellulose, methyl cellulose, hydroxyethyl cellulose, ethyl hydroxyethyl cellulose, hydroxypropyl cellulose (HPC); microcrystalline cellulose, silicon-modified microcrystalline cellulose (e.g. Prosolv®), guar flour, alginic acid and/or alginates; synthetic polymers such as polyvinyl pyrrolidone (povidone), polyvinyl acetate (PVAC), polyvinyl alcohol (PVA), polymers of acrylic acid and their salts, polyacrylamide, polymethacrylates, vinyl pyrrolidone/vinyl acetate copolymers (such as Kollidon® VA64, BASF), polyalkylene glycols, such as polypropylene glycol or preferably polyethylene glycol, co-block polymers of polyethylene glycol, especially co-block polymers of polyethylene glycol and polypropylene glycol (Pluronic®, BASF), and mixtures of the polymers mentioned. In addition, starch, starch derivatives, treated starch and pregelatinised starch can be used as adhesion promoters.

Substances particularly preferably used as adhesion promoters are polyvinyl pyrrolidone, preferably with a weight-average molecular weight of 10,000 to 60,000 g/mol, especially 12,000 to 40,000 g/mol, a copolymer of vinyl pyrrolidone and vinyl acetate, especially with a weight-average molecular weight of 40,000 to 70,000 g/mol and/or polyethylene glycol, especially with a weight-average molecular weight of 2,000 to 10,000 g/mol, and HPMC, especially with a weight-average molecular weight of 20,000 to 90,000 g/mol and/or preferably a content of methyl groups of 10 to 35% and a content of hydroxy groups of 1 to 35%. In addition microcrystalline cellulose can preferably be used, especially one with a specific surface area of 0.7-1.4 m²/g. The specific surface area is determined by means of the gas adsorption method according to Brunauer, Emmet and Teller. Finally, it is preferable to use pregelatinised starch, especially in formulations with a content of active agent of more than 70% by weight.

In addition, the adhesion promoter also includes solid, non-polymeric compounds which preferably contain polar side groups. Examples of these are sugar alcohols or disaccharides. Examples of suitable sugar alcohols and/or disaccharides are lactose, mannitol, sorbitol, xylitol, isomalt, glucose, fructose, maltose and mixtures thereof. The term “sugar alcohols” in this context also includes monosaccharides. Lactose and mannitol in particular are used as adhesion promoters.

Similarly, mixtures of the above-mentioned adhesion promoters are possible.

In preferred embodiments of the present invention, retigabine and adhesion promoter are used in an amount in which the weight ratio of retigabine to adhesion promoter is 20:1 to 1:10, more preferably 10:1 to 1:5, even more preferably 5 : 1 to 1 : 2, especially 4:1 to 2:1.

It is advantageous for the adhesion promoter to be used in particulate form and for a volume-average particle size (D50) of the adhesion promoter to be less than 500 μm, preferably 5 to 200 μm.

In one embodiment of the present invention, retigabine is used in micronised form.

The expression “micronised retigabine” is used in the context of this invention to denote particulate retigabine, which generally has an average particle diameter of 0.1 to 200 μm, preferably 0.5 to 100 μm, more preferably 1 to 50 μm, particularly preferably 1.5 to 25 μm and especially 2 μm to 10 μm.

The expression “average particle diameter” always relates in the context of this invention to the D50 value of the volume-average particle diameter determined by means of laser diffractometry. In particular, a Malvern Instruments Mastersizer 2000 was used to determine the diameter (wet measurement with ultrasound for 60 sec., 2,000 rpm, the evaluation being performed using the Fraunhofer model), and preferably using a dispersant in which the substance to be measured does not dissolve at 20° C.). The average particle diameter, which is also referred to as the D50 value of the integral volume distribution, is defined in the context of this invention as the particle diameter at which 50% by volume of the particles have a smaller diameter than the diameter which corresponds to the D50 value. Similarly, 50% by volume of the particles then have a larger diameter than the D50 value. The terms “average particle size” and “average particle diameter” are used synonymously in the context of this application.

The process of the invention can generally be carried out in two embodiments, namely as a dry-granulation process and as a direct-compression process. Both embodiments are carried out in the absence of solvent.

One aspect of the present invention therefore relates to a dry-granulation process comprising the steps of
(a) mixing retigabine with an adhesion promoter and optionally further pharmaceutical excipients;
(b) compacting it into a slug;
(c) granulating the slug;
(d) compressing the resulting granules into tablets, optionally with the addition of further pharmaceutical excipients; and
(e) optionally film-coating the tablets.

In step (a), retigabine and adhesion promoter and optionally further pharmaceutical excipients (described below) are mixed. The mixing can be performed in conventional mixers. The mixing may, for example, be performed in compulsory mixers or free-fall mixers, e.g. using a Turbula T 10B (Bachofen AG, Switzerland). Alternatively, it is possible that the retigabine is initially only mixed with part of the excipients (e.g. 50 to 95%) before compacting (b), and that the remaining part of the excipients is added after the granulation step (c). In the case of multiple compacting, the excipients should preferably be mixed in before the first compacting step, between multiple compacting steps or after the last granulation step.

The mixing conditions in step (a) and/or the compacting conditions in step (b) are usually selected such that at least 30% of the surface of the resulting retigabine particles-is- covered with adhesion promoter, more preferably at least 50% of the surface, particularly preferably at least 70% of the surface, especially at least 90% of the surface.

In step (b) of the method of the invention, the mixture from step (a) is compacted into a slug. It is preferable here that it should be dry compacting, i.e. the compacting is preferably performed in the absence of solvents, especially in the absence of organic solvents.

The compacting is preferably carried out in a roll granulator.

The rolling force is preferably 5 to 70 kN/cm, preferably 10 to 60 kN/cm, more preferably 15 to 50 kN/cm.

The gap width of the roll granulator is, for example, 0.8 to 5 mm, preferably 1 to 4 mm, more preferably 1.5 to 3 mm, especially 1.8 to 2.8 mm.

The compacting apparatus used preferably has a cooling means. In particular, the cooling is such that the temperature of the compacted material does not exceed 50° C., especially 40° C.

In step (c) of the process, the slug is granulated. The granulation can be performed with methods known in the state of the art. A Comill·U5 apparatus (Quadro Engineering, USA), for example, is used for granulating.

In a preferred embodiment, the granulation conditions are selected such that the resulting particles (granules) have a volume-average particle size ((D₅₀) value) of 50 to 800 μm, more preferably 100 to 750 μm, even more preferably 150 to 500 μm, especially 200 to 450 μm.

In addition, the granulation conditions can be selected such that no more than 55% of the particles are less than 200 μm in size or that the average particle diameter (D50) is between 100 and 450 μm.

In addition, the granulation conditions are preferably selected such that the resulting granules have a bulk density of 0.2 to 0.85 g/ml, more preferably 0.3 to 0.8 g/ml, especially 0.4 to 0.7 g/ml. The Hausner factor is usually in the range from 1.02 to 1.3, more preferably from 1.04 to 1.20 and especially from 1.04 to 1.15. The “Hausner factor” in this context means the ratio of tapped density to bulk density.

In a preferred embodiment, the granulation is performed in a screen mill. In this case, the mesh width of the screen insert is usually 0.1 to 5 mm, preferably 0.5 to 3 mm, more preferably 0.75 to 2 mm, especially 0.8 to 1.8 mm.

In a preferred embodiment, the process is adapted such that multiple compacting occurs, with the granules resulting from step (c) being returned once or more times to the compacting (b). The granules from step (c) are preferably returned 1 to 5 times, especially 2 to 3 times.

The granules resulting from step (c) can be further processed into pharmaceutical dosage forms. For this purpose, the granules are filled into sachets or capsules, for example. A subject matter of the invention is therefore also capsules and sachets containing a granulated pharmaceutical composition which is obtainable by the dry-granulation process of the invention.

The granules resulting from step (c) are preferably pressed into tablets (=step (d) of the process of the invention).

In step (d), compression into tablets occurs. Compression can be performed with tableting machines known in the state of the art. The compression is preferably performed in the absence of solvents.

Examples of suitable tableting machines are eccentric presses or rotary presses. As an example, a Fette® 102i (Fette GmbH, Germany) can be used. In the case of rotary presses, a compressive force of 2 to 40 kN, preferably 2.5 to 35 kN, is usually applied.

In step (d) of the process, pharmaceutical excipients may optionally be added to the granules from step (c). The amounts of excipients added in step (d) usually depend on the type of tablet to be produced and the amount of excipients which have already been added in steps (a) or (b).

In the optional step (e) of the process of the invention, the tablets from step (d) are film-coated. For this purpose, the methods of film-coating tablets which are standard in the state of the art can be employed.

For film-coating, macromolecular substances are preferably used, such as modified celluloses, polymethacrylates, polyvinyl pyrrolidone, polyvinyl acetate phthalate, zein and/or shellack or natural gum, such as carrageenan.

HPMC is preferably used, especially HPMC with a weight-average molecular weight of 10,000 to 150,000 g/mol and/or an average degree of substitution of —OCH₃ groups of 1.2 to 2.0.

The thickness of the coating is preferably 2 to 100 μm, especially 5 to 50 μm.

In addition to the dry-compacting and granulation processes described above, another aspect of the present invention is a compacted intermediate containing retigabine. An intermediate obtainable by jointly dry-compacting retigabine with an adhesion promoter is therefore a further subject matter of the invention.

As regards the properties of the retigabine to be used and the adhesion promoter to be used, reference may be made to the above explanations. The intermediate of the invention can be produced by steps (a) and (b) of the process of the invention explained above.

The compacting conditions for preparing the intermediate of the invention are usually selected such that the intermediate of the invention is present in the form of compacted material (a slug), the density of the intermediate being 0.8 to 1.3 g/cm³, preferably 0.9 to 1.20 g/cm³, especially 1.01 to 1.15 g/cm³.

The term “density” here preferably relates to the “pure density” (i.e. not to the bulk density or tapped density). The pure density can be determined with a gas pycnometer. The gas pycnometer is preferably a helium pycnometer; in particular, the AccuPyc 1340 helium pycnometer from the manufacturer Micromeritics, Germany, is used.

It is preferable that that the type and quantity of the adhesion promoter should be selected such that the resulting intermediate has a glass transition temperature (Tg) of more than 20° C., preferably >30° C.

It is preferable that the type and quantity of the adhesion promoter should be selected such that the resulting intermediate is storage-stable. “Storage-stable” means that in the intermediate of the invention, after storage for 3 years at 25° C. and 50% relative humidity, the proportion of crystalline retigabine—based on the total amount of retigabine—is no more than 60% by weight, preferably no more than 30% by weight, more preferably no more than 15% by weight, in particular no more than 5% by weight.

All the above remarks on the intermediate of the invention also apply to the product of the process resulting in step (b).

As described above under step (c) of the process of the invention, the intermediates of the invention may be comminuted, e.g. granulated. Normally, the intermediates of the invention are present in particulate form and have an average particle diameter (D50) of 1 to 750 μm, preferably von 1 to 350 μm, depending on the preparation method in each case.

The intermediate of the invention is usually employed to prepare a pharmaceutical formulation. For this purpose, in one embodiment, the intermediate—optionally together with further excipients (see explanations below)—is filled into single-dose and multiple-dose containers, preferably sachets and stick-packs. Single-dose and multiple-dose containers, preferably sachets and stick-packs containing the granules of the invention, are therefore also a subject matter of the invention.

As described above under step (d) of the process of the invention, the intermediate of the invention is preferably compressed into tablets in a further embodiment.

In the case of direct compression, only steps (a) and (d) and optionally (e) of the process described above are performed. One subject matter of the invention is therefore a process comprising the steps of
(a) mixing retigabine with an adhesion promoter and optionally further pharmaceutical excipients; and
(d) directly compressing the resulting mixture into tablets, and then
(e) optionally film-coating the tablets.

In principle, the explanations provided above on steps (a), (d) and (e) also apply to direct compression.

In a preferred embodiment, in the case of direct compression, step (a) includes jointly milling retigabine and adhesion promoter. Further pharmaceutical excipients may optionally be added.

The milling conditions are usually selected such that at least 30% of the surface of the resulting retigabine particles is covered with adhesion promoter, more preferably at least 50% of the surface, particularly preferably at least 70% of the surface, especially at least 90% of the surface.

The milling is generally performed in conventional milling apparatuses, such as in a ball mill, air jet mill, pin mill, classifier mill, cross-beater mill, disk mill, mortar grinder, rotor mill. The milling time is usually 0.5 minutes to 1 hour, preferably 2 minutes to 50 minutes, more preferably 5 minutes to 30 minutes.

In the case of direct compression, it is preferable that in step (d), a mixture is used in which the particle sizes of the active agent and the excipients are matched to one another. Preferably, retigabine, adhesion promoter and, where applicable, any further pharmaceutical excipients are used in particulate form with an average particle size (D50) of 35 to 250 μm, more preferably 50 to 200 1.1M, especially 70 to 150 μm.

Both in the case of dry granulation and in the case of direct compression, further pharmaceutical excipients may be used in addition to retigabine and adhesion promoter. These are the excipients with which the person skilled in the art is familiar, especially those which are described in the European Pharmacopoeia. The same applies to the use of the intermediate of the invention for filling into single-dose and multipledose containers.

Examples of excipients used are disintegrants, anti-stick agents, emulsifiers, pseudoemulsifiers, fillers, additives to improve the powder flowability, glidants, wetting agents, gel-forming agents and/or lubricants. Where appropriate, further excipients can also be used.

“Disintegrants” is the term generally used for substances which accelerate the disintegration of a dosage form, especially a tablet, after it is placed in water. Suitable disintegrants are, for example, organic disintegrants such as carrageenan, croscarmellose and crospovidone. Alkaline disintegrants are likewise used. The term “alkaline disintegrants” means disintegrants which, when dissolved in water, produce a pH level of more than 7.0.

Inorganic alkaline disintegrants are preferably used, especially salts of alkali and alkaline earth metals. Preferred examples here are sodium, potassium, magnesium and calcium. As anions, carbonate, hydrogen carbonate, phosphate, hydrogen phosphate and dihydrogen phosphate are preferred. Examples are sodium hydrogen carbonate, sodium hydrogen phosphate, calcium hydrogen carbonate and the like.

The formulation of the invention optionally contains fillers. “Fillers” are generally understood to mean substances which serve to form the body of the tablet in the case of tablets with small amounts of active agent (e.g. less than 70% by weight). This means that fillers “dilute” the active agents in order to produce an adequate tablet-compression mixture. The normal purpose of fillers, therefore, is to obtain a suitable tablet size.

Examples of preferred fillers are talcum, calcium phosphate, sucrose, calcium carbonate, magnesium carbonate, magnesium oxide, maltodextrin, calcium sulphate, dextrates, dextrin, dextrose, hydrogenated vegetable oil, kaolin, sodium chloride, and/or potassium chloride.

Fillers are usually employed in the present case in a small amount, since a high content of active agent is desirable. Fillers may, for example, be used in an amount of 0 to 20% by weight, more preferably 0 to 10° A) by weight, based on the total weight of the formulation.

One example of an additive to improve the powder flowability is disperse silicon dioxide, e.g. known under the trade name Aerosil·.

Additives to improve the powder flowability are usually employed in an amount of 0.1 to 3% by weight, based on the total weight of the formulation.

In addition, lubricants may be used. Lubricants are generally used in order to reduce sliding friction. In particular, the intention is to reduce the sliding friction found during tablet pressing between the punches moving up and down in the die and the die wall, on the one hand, and between the edge of the tablet and the die wall, on the other hand. Suitable lubricants are, for example, stearic acid, adipic acid, sodium stearyl fumarate and/or magnesium stearate.

Lubricants are normally used in an amount of 0.1 to 5% by weight, preferably 0.5 to 3% by weight, based on the total weight of the formulation.

It lies in the nature of pharmaceutical excipients that they sometimes perform more than one function in a pharmaceutical formulation. In the context of this invention, in order to provide an unambiguous delimitation, the fiction will therefore preferably apply that a substance which is used as a particular excipient is not simultaneously also used as a further pharmaceutical excipient.

The ratio of active agent to excipients is preferably selected such that the formulations resulting from the process of the invention (i.e. the tablets of the invention for example) contain 35 to 90% by weight, more preferably 55 to 85% by weight, especially 60 to 80% by weight retigabine and 10 to 65% by weight, more preferably 15 to 45% by weight, especially 20 to 40% by weight pharmaceutically acceptable excipients.

In these ratios specified, the amount of adhesion promoter used in the process of the invention or used to prepare the intermediate of the invention is counted as an excipient. This means that the amount of active agent refers to the amount of retigabine contained in the formulation.

It has been shown that the formulations of the invention (i.e. the tablets of the invention or the granules of the invention which result from step (c) of the process of the invention and which can be filled into stick-packs or sachets, for example) may serve both as a dosage form with immediate release (or “IR” for short) and also as a dosage form with modified release (or “MR” for short).

In a preferred embodiment for an IR formulation, especially in the form of a peroral tablet, a relatively large amount of disintegrant is used. In that preferred embodiment, the pharmaceutical formulation of the invention therefore contains 1 to 30% by weight, more preferably 3 to 15% by weight, especially 5 to 12% by weight disintegrants, based on the total weight the formulation.

In a preferred embodiment for an MR formulation, especially in the form of a peroral tablet, a relatively small amount of disintegrant is used. In that preferred embodiment, the pharmaceutical formulation of the invention therefore contains 0.1 to 10% by weight, more preferably 0.5 to 8% by weight, especially 1 to 5% by weight disintegrants, based on the total weight the formulation.

In the case of the MR formulation, croscarmellose or crospovidone is preferred as the disintegrant. In the case of the IR formulation, alkaline disintegrants are preferred.

In addition the conventional retardation techniques can be used for the MR formulation.

The above-mentioned pharmaceutical excipients can be used in both the preferred embodiments (dry granulation and direct compression). Furthermore, the tableting conditions in both embodiments of the process of the invention are preferably selected such that the resulting tablets have a ratio of tablet height to weight of 0.005 to 0.3 mm/mg, particularly preferably 0.05 to 0.2 mm/mg.

The process of the invention is preferably performed such that the tablets of the invention contain retigabine in an amount of more than 200 mg to 1,000 mg, more preferably 250 mg to 900 mg, especially 300 mg to 600 mg. The subject matter of the invention thus relates to tablets containing 300 mg, 400 mg, 450 mg, 600 mg or 900 mg retigabine.

In addition, the resulting tablets preferably have a hardness of 50 to 300 N, particularly preferably 80 to 250 N, especially 100 to 220 N. The hardness is determined in accordance with Ph. Eur. 6.0, section 2.9.8.

Also, the resulting tablets preferably have a friability of less than 3%, particularly preferably less than 2%, especially less than 1%. The friability is determined in accordance with Ph. Eur. 6.0, section 2.9.7.

Finally, the tablets of the invention usually have a “content uniformity” of 95 to 105% of the average content, preferably 98 to 102%, especially 99 to 101%. (This means that all the tablets have a content of active agent of between 95 and 105%, preferably between 98 and 102%, especially between 99 and 101% of the average content of active agent.) The “content uniformity” is determined in accordance with Ph. Eur. 6.0, section 2.9.6.

In the case of an IR formulation, the release profile of the tablets of the invention after 10 minutes according to the USP method usually indicates a content released of at least 30%, preferably at least 60%, especially at least 90%.

In the case of an MR formulation, the release profile of the tablets of the invention after 60 minutes according to the USP method usually indicates a content released of 10%, preferably 20%, especially 30%.

The above details regarding hardness; friability, content uniformity and release profile preferably relate here to the non-film-coated tablet for an IR formulation. For a modified-release tablet, the release profile relates to the total formulation.

The tablets produced by the process of the invention may be tablets which can be swallowed unchewed (non-film-coated or preferably film-coated). They may likewise be dispersible tablets. “Dispersible tablet” here means a tablet to be used for producing an aqueous suspension for swallowing.

In the case of tablets which are swallowed unchewed, it is preferable that they be coated with a film layer, as explained above under step (e). The above-mentioned ratios of active agent to excipient, however, relate to the uncoated tablet.

For sachets, the following excipients are usually employed:

In a preferred embodiment, the granules described above (intermediate) are additionally mixed, after dry processing, with one or more flavourings, e.g. peppermint flavour. The proportion of flavourings is usually 0.1-10% by weight, preferably 2-4% by weight, based on the total weight of the formulation. In the context of this application, the term “flavourings” is to be understood as defined in Council Directive 88/388/EWG of 22nd June, 1988. Instead of the peppermint flavour, it is also possible to use other flavours, such as spearmint, lemon or something like that.

In addition, in a preferred embodiment, the intermediate is mixed with a sweetener, the sweetener preferably being used in an amount of 0.1 to 4, more preferably 1 to 3% by weight based on the total weight of the formulation. Saccharine sodium, for example, is particularly suitable for this purpose, e.g. in a concentration of 1-3% by weight. Aspartam can also be used in a concentration of 2-4% by weight. Apart from that, other standard sweeteners can also be used.

The sachet formulation may also contain an effervescent element consisting of a mixture of citric acid and sodium hydrogen carbonate, e.g. in a ratio of 1:2, and preferably accounts for 5 to 15% by weight, such as 10% by weight, of the total amount.

As explained above, the subject matter of the invention is not only the process of the invention, but also the tablets produced with that process. It has further been found that the tablets produced with this process preferably have a bimodal pore size distribution. One subject matter of the invention is thus tablets containing retigabine or a pharmaceutically acceptable salt thereof and adhesion promoter and optionally pharmaceutically acceptable excipients, wherein the tablets have a bimodal pore size distribution.

This tablet of the invention is formed when the granules from process step (c) are compressed. This compressed material-consists of_solid and pores. The pore structure can be characterised more specifically by determining the pore size distribution.

The pore size distribution was determined by means of mercury porosimetry. Mercury porosimetry measurements were made with the Micromeritics, Norcross, USA, “Poresizer” porosimeter. The pore sizes were calculated assuming a mercury surface tension of 485 mN/m. The cumulative pore volume was used to calculate the pore size distribution as the cumulative frequency distribution or proportion of the pore fractions in per cent. The average pore diameter (4V/A) was determined from the total specific mercury intrusion volume (Vgesᵢₙₜ) and the total pore surface area (Agespₚₒᵣ) according to the following equation.

$$4 V / A = \frac{4 \cdot {Vges}_{int} [ ml \text{/} g ]}{{Ages}_{por} [ {m}^{2} \text{/} g ]}$$

“Bimodal pore size distribution” is understood to mean that the pore size distribution has two maxima. The two maxima are not necessarily separated by a minimum, but rather a head and shoulders pattern is also regarded as bimodal for the purposes of the invention.

The formulations of the invention are preferably used for the treatment of epilepsy and neuropathic pain. One subject matter of the invention is thus the use of dry-compacted retigabine for the treatment of epilepsy and neuropathic pain.

The invention will now be explained with reference to the following examples.

### EXAMPLES

In all the Examples, retigabine is preferably used in the form of retigabine dihydrochloride, the amount specified referring to the amount of retigabine in'the form of the free base. This means that the statement of 300 mg retigabine corresponds to about 372 mg retigabine dihydrochloride.

### Example 1a

### Direct Compression of Retigabine (IR)

| | |
|---|---|
| Retigabine | 400 mg |
| Lactose monohydrate | 200 mg |
| Microcrystalline cellulose | 45 mg |
| Magnesium stearate | 5 mg |
| Silica | 1 mg |
| Croscarmellose | 5 mg |

Retigabine together with lactose was premixed for 10 minutes in a free-fall mixer (Turbula). After that, all the other ingredients except for magnesium stearate were added and mixed for a further 30 min. After the addition of magnesium stearate, final mixing continued for 2 min. The finished mixture was compressed on a rotary tableting press with punches with the following properties: 10 mm round biconvex. The tablets had a hardness of approx. 110 N auf. After that, the tablets could optionally be covered with a coating.

For the film-coating, hypromellose (Pharmacoat 603) and polyethylene glycol 6,000 were mixed with water, and, after they had dissolved, combined with a separately prepared suspension of titanium dioxide and iron oxide in water. The tablet cores were coated with the resulting suspension in a perforated-drum coater.

### Example 1b

Example 1a was repeated, using 10 mg magnesium stearate.

### Example 2a

### Dry Granulation of Retigabine and Compression into Tablets (IR)

| | |
|---|---|
| Retigabine | 300 mg |
| Povidone VA64 | 300 mg |
| Prosolv (Si-modified microcryst. cellulose) | 80 mg |
| Sodium bicarbonate | 40 mg |
| Magnesium stearate | 4 mg |
| Silica | 4 mg |

Retigabine was premixed together with povidone VA64, sodium bicarbonate and 50% by weight Prosolv·, magnesium stearate and silica and then compacted (rolling force 30 kN/cm, gap width 2 mm). The resulting intermediate was granulated using a screen mill (1.0 mm) and compressed into tablets with the remaining Prosolw, magnesium stearate and silica.

It was possible to film-coat the resulting tablets (as described in Example 1a).

### Example 2b

Example 2a was repeated, using 8 mg magnesium stearate.

### Example 3a

### Direct Compression of Retigabine (SR)

| | |
|---|---|
| Retigabine | 600 mg |
| Pharmacoat ® 603 (retarding hydroxypropyl | 300 mg |
| methyl cellulose) | |
| Prosolv ® | 50 mg |
| Talcum | 4 mg |
| Magnesium stearate | 4 mg |

Retigabine was mixed with hydroxypropyl methyl cellulose and Prosolva for (Turbula T10B, 30 minutes). Magnesium stearate and talcum were added to the mixture, and everything together was mixed for a further 3 minutes.

The finished mixture was pressed into tablets. For this purpose, an eccentric press, Korsch EKO, was used with a pressing a force of 10 kN.

### Example 3b

Example 3a was repeated, using 8 mg magnesium stearate.

### Example 4a

### Direct Compression of Retigabine (IR)

| | |
|---|---|
| Retigabine | 900 mg |
| Pregelatinised starch | 200 mg |
| Magnesium stearate | 5 mg |

The active agent was mixed together with the starch in the free-fall mixer (Turbula W10B) for 20 minutes, after which the magnesium stearate was added.

The finished mixture was mixed again for 3 minutes and then compressed on an eccentric press (Korsch, EKO).

### Example 4b

Example 4a was repeated, using 10 mg magnesium stearate.

### Example 5

### Sachet Containing Dry-Granulated Intermediate

| | |
|---|---|
| Retigabine | 900 mg |
| Microcrystalline cellulose | 200 mg |
| Polyvinyl pyrrolidone | 100 mg |
| Saccharine sodium | 150 mg |
| Peppermint flavour | 40 mg |
| Citric acid | 50 mg |
| Sodium bicarbonate | 100 mg |

Retigabine was mixed with sodium bicarbonate, microcrystalline cellulose, polyvinyl pyrrolidone, citric acid and peppermint flavour and then compacted (40 kN, 2 mm). The compacted material was screened to a particle size of 0.8 mm through a screen accompanied by comminution (Comil® U5). The granules obtained were filled into sachets.

### Example 6

### Effervescent Tablet

| | |
|---|---|
| Retigabine | 900 mg |
| Microcrystalline cellulose | 200 mg |
| Polyvinyl pyrrolidone | 200 mg |
| Peppermint flavour | 40 mg |
| Citric acid | 50 mg |
| Sodium bicarbonate | 100 mg |
| Magnesium stearate | 5 mg |
| Aerosil ® | 3 mg |

The ingredients, apart from the lubricant, were dry-mixed (Turbula W 10B) and screened (710 μm). The magnesium stearate was added and the finished mixture was compressed into an effervescent tablet.

1. A process for the production of an oral dosage form containing comprising retigabine and an adhesion promoter, wherein the dosage form is produced by of dry compacting or by direct compression, and wherein the oral dosage form comprises tablets.

2. The process as claimed in claim 1, comprising the steps of
(a) mixing retigabine with an the adhesion promoter and optionally further pharmaceutical excipients;
(b) compacting it into a slug;
(c) granulating the slug; and
(d) compressing the resulting granules into tablets, optionally with the addition of further pharmaceutical excipients; and

3. The process as claimed in claim 2, wherein the compacting (b) is performed in a roll compacter and the rolling force is 5 to 70 kN/cm, preferably 10 to 50 kN/cm.

4. The process as claimed in claim 2, wherein the granulation conditions in step (c) are selected such that no more than 55% of the particles are less than 200 μm in size or that the average particle diameter (D50) is between 100 and 450 μm.

5. The process as claimed in claim 1, comprising the steps of
(a) mixing retigabine with an the adhesion promoter and optionally further pharmaceutical excipients; and
(d) directly compressing the resulting mixture into tablets, and

6. The process as claimed in claim 5, wherein step (a) includes jointly milling retigabine and the adhesion promoter.

7. The process as claimed in claim 5, wherein in step (d), a mixture of retigabine, the adhesion promoter and optionally further pharmaceutical excipients with an average particle size (D50) of 50 to 250 μm is used.

8. The process as claimed in claim 1, wherein retigabine is used in an amount of 55 to 90% by weight, based on the total weight of all the substances used.

9. Tablets produced by a process as claimed in claim 1.

10. The tablets as claimed in claim 9 with a friability of less than 3%, a content uniformity of 95 to 105% and a hardness of 50 to 250 N, wherein the tablets contain 250 to 900 mg retigabine.

11. An intermediate produced by jointly dry-compacting retigabine with an adhesion promoter.

12. The intermediate as claimed in claim 10, wherein the density of the intermediate is 0.8 to 1.3 g/cm³.

13. The intermediate as claimed in claim 10, wherein the adhesion promoter used is a polymer with a weight-average molecular weight of less than 90,000 g/mol and a glass transition temperature (Tg) of more than 20° C. after being heated up twice or a sugar alcohol is used.

14. The intermediate as claimed in claim 10, wherein the weight ratio of retigabine to adhesion promoter is 5:1 to 1:2.

15. A sachet or stick-pack comprising an intermediate as claimed in claim 11.

16. The use of dry-compacted retigabine for the treatment of epilepsy and neuropathic pain.

17. The process as claimed in claim 3, wherein the compacting (b) is performed in a roll compacter and the rolling force is10 to 50 kN/cm.

18. The intermediate as claimed in claim 12, wherein the density of the intermediate is 0.9 to 1.20 g/cm³.

19. The process as claimed in claim 2, further comprising the step of (e) film-coating the tablets.

20. The process as claimed in claim 5, further comprising the step of (e) film-coating the tablets.
